(19) United States
(12) Patent Application Publication
(10) Pub. No.: US 2022/0323534 A1
(43) Pub. Date: Oct. 13, 2022

(54) USE OF A COMPOSITION CONTAINING AN EXTRACT OF TULIPA GESNERIANA

(71) Applicant: FRANCE COSMEPHYL-LAB, Paris (FR)

(72) Inventors: Michel HOCQUAUX, Paris (FR); Jean-Christophe AUBAGNAC, Saint Jean (FR)

(73) Assignee: FRANCE COSMEPHYL-LAB, Paris (FR)

(21) Appl. No.: 17/640,451

(22) PCT Filed: Sep. 4, 2020
(86) PCT No.: PCT/EP2020/074824

§ 371 Date: Mar. 4, 2022

(30) Foreign Application Priority Data
Sep. 4, 2019 (FR) FR1909730

(51) Int. Cl. A61K 36/896; A61K 38/07; A61K 8/42; A61K 8/73; A61Q 19/08

(52) U.S. Cl. CPC A61K 36/896; A61K 38/07; A61K 8/42; A61K 8/735; A61K 8/738; A61Q 19/08

(57) ABSTRACT

New compositions containing an extract of Tulipa Gesneriana. The compositions include a tulip extract from the Tulipa Gesneriana family, acetyl-tetrapeptide-2, and acetyl-hexapeptide-51 amide. Also, the use thereof in cosmetics, and the use thereof in pharmaceuticals, particularly in dermatology for treating or preventing skin aging, and the use thereof in pharmaceuticals, particularly in dermatology, for treating or preventing pathologies linked to oxidative stress.

The present invention relates to new compositions comprising an extract of Tulipa Gesneriana, their use in cosmetics, as well as their pharmaceutical use, in particular in dermatology.

Skin aging is often described as the result of two mechanisms.

The first concerns aging due to external factors such as the sun, pollution or lifestyle, which lead to the formation of free radicals. Free radicals are “waste” molecules produced during cell respiration.

Normally eliminated as they are produced, they can be found in too high a quantity, and exceed the cleaning capacities of the cells. This situation where the cell is “overwhelmed” and can no longer suppress free radicals is called “oxidating stress” or “oxidative stress”. Free radicals attack and degrade the interior of cells as well as the environment in which they live, causing damage to living tissue. The skin is an organ particularly exposed to oxidative stress. The sun's UV radiation can, in contact with the lipids of the epidermis, form free radicals. All skin structures are subject to oxidative stress, resulting in accelerated skin aging with the appearance of dark spots, fine lines, loss of elasticity and density.

The second is “chronological” aging. Chronological cutaneous aging is a complex phenomenon, which can be considered as genetically programmed, with however a predominant harmful action of the oxygenated free radicals generated in the organism in an intrinsic way. It results clinically in atrophy of the skin with cutaneous fragility, loss of elasticity, an accentuation of expression lines and functional modifications with a reduction in the immune and inflammatory response. Histologically, chronological aging results in a decrease in the thickness of the epidermis with flattening of the dermo-epidermal junction, dermal atrophy with a decrease in collagen, elastolysis of elastic fibers and an alteration of microcirculation.

There is a continuing demand for compositions, especially topical ones, in the fight against skin aging and oxidative stress. Compositions based on natural products, including for example plant extracts, or biomimetic products, inspired by nature, are particularly advantageous.

Recently, extracts of Tulipa Gesneriana have been isolated and used in cosmetic compositions.

A purple tulip extract has notably been developed for use on the hair. However, Tulip extracts have been little used in anti-aging skin applications.

One of the aims of the invention is to provide a new combination comprising a tulip extract.

Another object is to provide a new cosmetic composition comprising a tulip extract.

Another object is to provide a new pharmaceutical composition comprising a tulip extract.

Another object of the invention is to be able to use the new combination, or a composition comprising it, in the treatment or prevention of skin aging.

Another object of the invention is to be able to use the new combination, or a composition comprising it, in the treatment or prevention of pathologies linked to oxidative stress.

A sixth object is to provide a cosmetic treatment method using the new composition.

A first object of the present invention is a combination comprising or consisting of the following three ingredients:
an extract of tulip of the species of Tulipa Gesneriana, in particular an extract of purple tulip, and
acetyl-tetrapeptide-2, and
acetyl-hexapeptide-51 amide.

The combination according to the present invention is based on a natural ingredient, derived from a flower. Indeed, it is a tulip extract, brought together with two peptides. Both peptides are “biomimetic peptides” that mimic the action of natural peptides, while being biocompatible.

The synergy between the three ingredients gives rise to an combination whose use in a cosmetic or pharmaceutical composition confers interesting properties, in particular in the fight against skin aging and the correction of skin imperfections.

Indeed, the associated ingredients present a remarkable synergy to protect and restore the barrier function, fight against the effects of oxidative stress and have an action on various genes involved in aging.

Tulipa Gesneriana is a species of tulip belonging to the Liliaceae family.

The purple tulip is a variety belonging to the genus Tulipa Gesneriana.

This is a purple/black colored tulip which is also known as the “black tulip” or “queen of the night” and whose flowers are particularly rich in flavonoids.

“Tulip extract” means an extract obtained from the plant and/or part of the plant.

It may be, for example, an extract obtained from flowers or flowering tops, leaves, seeds, roots, stems, tulip pericarp, preferably tulip petals.

The tulip extract used in the present invention is preferably in the form of a powder.

The purple tulip extract that can be used in the present invention is contained in a product commercially available under the name Dumaflorine® from the company Greenpharma. It is a purple tulip extract formulated with Maltodextrin.

By “acetyl tetrapeptide-2” is meant a tetrapeptide of sequence “Ac—Lys—Asp—Val—Tyr—OH” obtained by acylation of tetrapeptide-2.

The structure of the molecule, whose CAS number is [757942-88-4] is shown below.

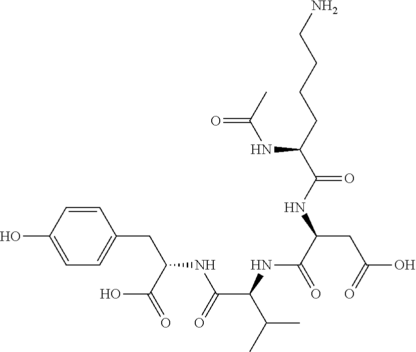 [SMILES: CC(=O)N[C@@H](CCCCN)C(=O)N[C@@H](CC(=O)O)C(=O)N[C@H](C(=O)N[C@@H](Cc1ccc(O)cc1)C(=O)O)C(C)C]

### Chemical Structure of Acetyl Tetrapeptide-2

The molecule is commercially available in several forms that can be used in the context of the present invention.

The Uplevity™ product from Lipotec, for example, is a solution of acetyl-tetrapeptide-2 in a Water/Caprylyl Glycol mixture.

By “acetyl-hexapeptide-51 amide” is meant the acylated derivative of hexapeptide-51 in which the C-terminal is an amide.

The product is commercially available in forms that can be used in the context of the present invention.

The Juveleven™ product from Lipotec, for example, is a solution of acetyl-hexapeptide-51 amide in a water/butylene glycol mixture.

According to another particular embodiment, the present invention relates to a combination as defined above, in which the tulip extract is as obtained by a process comprising:
a step A of one or more macerations, to obtain a liquid extract,
a step B of clarification of the liquid extract to obtain a clarified liquid extract,
a step C of drying of the clarified liquid, to obtain the tulip extract.

Step A of maceration is notably carried out 1 to 3 times, in particular once. Said step A is preferably carried out by maceration of the flowers, in particular by maceration of the tulip petals, in an ethanol/water mixture, in particular in a solution of 30% ethanol in sterile water.

For example, 1 kg of tulip petals can be macerated for 24 hours, at room temperature, in 10 liters of a 30% ethanol solution in sterile water.

Clarification step B is preferably carried out by filtration, in particular by filtration on a filter having a porosity of 10 to 15 μm, optionally followed by a second filtration on a filter having a porosity of 0.15 to 0.25 μm.

The drying step C is in particular carried out by evaporation of the solvents under vacuum.

According to a particular embodiment, the subject of the present invention is a combination as defined above, in which the combination comprises hyaluronic acid whose molecular weight is between 20 and 50 kDa.

In other words, the present invention relates to a combination comprising or consisting of the following four ingredients:
an extract of tulip of the species of Tulipa Gesneriana, in particular an extract of purple tulip, and
acetyl-tetrapeptide-2, and
acetyl-hexapeptide-51 amide,
hyaluronic acid.

The term “from 20 to 50 kDa” also means the following ranges: 20 to 45 kDa, 20 to 40 kDa, 20 to 35 kDa, 20 to 30 kDa, 20 to 25 kDa, 25 to 50 kDa, 30 to 50 kDa, 35 to 50 kDa, 40 to 50 kDa, 45 to 50 kDa, 25 to 45 kDa, 30 to 40 kDa.

Hyaluronic acid is a macromolecule made up of a repetitive sequence of sugar units.

Indeed, the macromolecule is made up of disaccharides, composed of D-glucuronic acid and D-N-acetylglucosamine, linked together by alternating β-1,4 and β-1,3 glycosidic bonds. The polymer, whose CAS number is [9004-61-9], exists in a wide range of molecular masses, which can be between 4 and 8000 kDa.

The hyaluronic acid used in the present invention is a low molecular mass polymer, between 20 and 50 kDa.

The hyaluronic acid that can be used in the present invention can be in different forms, in particular in the form of microspheres.

Hyaluronic acid has important moisturizing and tissue supporting properties. The skin, which contains about 50% of the hyaluronic acid in the human body, is one of the richest organs in hyaluronic acid.

The presence of hyaluronic acid in the combination of the present invention leads to a synergistic effect between said hyaluronic acid and the three other ingredients. The synergy between the different ingredients gives rise to an combination whose use in a cosmetic or pharmaceutical composition confers interesting properties, in particular to protect and restore the barrier function, to fight against the effects of oxidative stress and to have an action on different genes involved in ageing.

According to another particular embodiment, the present invention relates to a combination as defined above, said combination being devoid of niacinamide, poppy seed oil, yeast extract, tamarindus indica seed gum, Cycnoches Cooperi (Orchid) Flower/Leaf Extract,

Amodimethicone, Vitamin E (a-tocopherol) or Baobab Seed Oil. According to another particular embodiment, the present invention relates to a combination as defined above, in which the combination comprises maltodextrin.

The commercial product under the name of Dumaflorine® from the company Greenpharma is formulated with maltodextrin.

The use of said commercial product therefore leads to a combination comprising maltodextrin.

The latter is a priori inert, and can be considered as an excipient.

According to another particular embodiment, the present invention relates to a combination comprising or consisting of the following three ingredients:
a tulip extract from the Tulipa Gesneriana family, in particular a purple tulip extract, and
acetyl-tetrapeptide-2, and
acetyl-hexapeptide-51 amide,
   and optionally hyaluronic acid whose molecular weight is between 20 and 50 kDa, and optionally maltodextrin.

According to another particular embodiment, the present invention relates to a combination as defined above comprising or consisting of the following four ingredients:
a tulip extract from the Tulipa Gesneriana family, in particular a purple tulip extract, and
acetyl-tetrapeptide-2, and
acetyl-hexapeptide-51 amide,
   hyaluronic acid whose molecular mass is between 20 and 50 kDa, and optionally maltodextrin.

According to another particular embodiment, the present invention relates to a combination as defined above, in which the tulip extract is formulated with maltodextrin. said formulation comprising tulip extract and maltodextrin is in particular as obtained by a process comprising:
a step A of one or more macerations, to obtain a liquid extract,
a step B of clarification of the liquid extract to obtain a clarified liquid extract,
a step C-2 of partial evaporation of the clarified liquid, to obtain a solution of the tulip extract.
a step D of addition of maltodextrin, to obtain a solution of tulip extract and maltodextrin,
a drying step D, to obtain a tulip extract formulated with maltodextrin.

Step A of maceration is notably carried out 1 to 3 times, in particular once. Said step A is preferably carried out by maceration of the flowers, in particular by maceration of the tulip petals, in an ethanol/water mixture, in particular in a solution of 30% ethanol in sterile water.

For example, 1 kg of tulip petals can be macerated for 24 hours, at room temperature, in 10 liters of a 30% ethanol solution in sterile water.

Clarification step B is preferably carried out by filtration, in particular by filtration on a filter having a porosity of 10 to 15 μm, optionally followed by a second filtration on a filter having a porosity of 0.15 to 0.25 μm.

The partial evaporation step C-2 is in particular carried out by vacuum evaporation.

For example, a clarified solution obtained from 1 kg of tulip petals, macerated in 10 liters of a 30% solution of ethanol in sterile water is evaporated to obtain 80 grams of a solution of tulip extract.

Step D is preferably carried out with an amount of maltodextrin comprised from 800 to 1000%, preferably 900% by weight relative to the weight of the tulip extract solution obtained at the end of step C-2. For example, 720 grams of maltodextrin is mixed with 80 grams of the tulip extract solution.

Step E is preferably carried out by spray drying.

The tulip extract included in the combination according to the present invention comprises in particular,
from 20 to 70% of sugars, and
from 15 to 30% of phenolic compounds, of which at least 8% of the phenolic compounds are flavonoids,
in percentage by weight relative to the total weight of the dry extract.

The sugars are in particular monosaccharides and polysaccharides.

The flavonoids comprise in particular from 50 to 90% quercetin and from 10 to 50% of kampferol, as a percentage by weight relative to the total weight of the flavonoids.

The flavonoids comprise in particular 65 to 85% quercetin and 15 to 35% of kampferol, as a percentage by weight relative to the total weight of the flavonoids.

According to another particular embodiment, the present invention relates to a combination as defined above, in which the tulip extract comprises,
from 20 to 70% of sugars, in particular monosaccharides and polysaccharides, and
from 15 to 30% of phenolic compounds, of which at least 8% of the phenolic compounds are flavonoids,
in percentage by weight relative to the total weight of the dry extract, said flavonoids notably comprise 50 to 90% of quercetin and 10 to 50% of kampferol, in particular 65 to 85% quercetin and 15 to 35% of kampferol
in percentage by weight relative to the total weight of the flavonoids.

According to another particular embodiment, the present invention relates to a combination as defined above, in which the quantity of the tulip extract, in particular of purple tulip, is greater than 0% and less than or equal to 5%, in particular comprised from 0.1 to 5%, as a percentage by weight relative to the total weight of the combination.

“From 0.1 to 5%” also means the following ranges: 0.1 to 4%, 0.1 to 3%, 0.1 to 2%, 0.1 to 1%, 0.5 to 5%, 1 to 5%, 2 to 5%, 3 to 5%, 4 to 5%, 0.2 to 4%, 0.5 to 3%, and 1 to 2%.

According to another particular embodiment, the present invention relates to a combination as defined above, in which the quantity of the tulip extract, in particular of purple tulip, is greater than 0% and less than or equal to 0.05%, in particular comprised from 0.001 to 0.05%, in particular from 0.001 to 0.04%, as a percentage by weight relative to the total weight of the combination.

“From 0.001 to 0.05%” also means the following ranges: 0.005 to 0.05%, 0.01 to 0.05%, 0.02 to 0.05%, 0.03 to 0.05% , 0.04 to 0.05%, 0.001 to 0.04%, 0.001 to 0.03%, 0.001 to 0.02%, 0.002 to 0.04%, 0.005 to 0.03%, 0.01 to 0 0.02%, and in particular about 0.015%.

The Dumaflorine® product from Greenpharma, advantageously used in an amount of 0.01 to 0.5%, as a percentage by weight relative to the total weight of the combination, or 0.05 to 0.5%, of 0.1 to 0.5%, 0.2 to 0.5%, 0.3 to 0.5%, 0.4 to 0.5%, 0.01 to 0.4%, 0.01 to 0.3%, from 0.01 to 0.4%, from 0.05 to 0.4%, from 0.1 to 0.3%, from 0.1 to 0.2 and in particular by about 0.15%.

According to another particular embodiment, the present invention relates to a combination as defined above, in which the quantity of acetyl-tetrapeptide-2 is between 0.005 and 0.05%, in percentage by weight relative to the weight total of the combination.

“From 0.005 to 0.05%” also means the following ranges: 0.005 to 0.04%, 0.005 to 0.03%, 0.005 to 0.02%, 0.005 to 0.01%, 0.01 to 0 0.5%, 0.02 to 0.05%, 0.03 to 0.05%, 0.04 to 0.05%, 0.01 to 0.04%, 0.02 to 0.03%.

According to another particular embodiment, the present invention relates to a combination as defined above, in which the quantity of acetyl-tetrapeptide-2 is between 0.025 and 0.25%, in particular from 0.05 to 0, 15%, preferably about 0.05%, as a percentage by weight relative to the total weight of the combination.

“From 0.025 to 0.25%” also means the following ranges: 0.025 to 0.05%, 0.25 to 0.1%, 0.025 to 0.15%, 0.025 to 0.20%, 0.04 to 0.25%, 0.05 to 0.25%, 0.075 to 0.25%, 0.10 to 0.25%, 0.15 to 0.25%, 0.20 to 0.25%, 0 0.04 to 0.20%, 0.05 to 0.2%, 0.05 to 0.15%, 0.075 to 0.10%, 0.09 to 0.10%, and in particular about 0.05% .

The Uplevity™ product from Lipotec is a solution comprising 0.05% acetyl-tetrapeptide-2, as a percentage by weight relative to the total weight of the commercial product.

A quantity of acetyl-tetrapeptide-2 between 0.00025 and 0.0025% is therefore equivalent to a quantity of Uplevity™ product between 0.5 and 5%.

According to another particular embodiment, the present invention relates to a combination as defined above, in which the amount of acetyl-hexapeptide-51 amide is between 0.005 and 0.05%, as a percentage by weight relative to the total weight of the combination.

“From 0.005 to 0.05%” also means the following ranges: 0.005 to 0.04%, 0.005 to 0.03%, 0.005 to 0.02%, 0.005 to 0.01%, 0.01 to 0 0.5%, 0.02 to 0.05%, 0.03 to 0.05%, 0.04 to 0.05%, 0.01 to 0.04%, 0.02 to 0.03%.

According to another particular embodiment, the present invention relates to a combination as defined above, in which the quantity of acetyl-hexapeptide-51 amide is between 0.025 and 0.25%, in particular from 0.05 to 0 2%, preferably about 0.1%, as a percentage by weight relative to the total weight of the combination.

“From 0.025 to 0.25%” also means the following ranges: 0.025 to 0.05%, 0.25 to 0.1%, 0.025 to 0.15%, 0.025 to 0.20%, 0.04 to 0.25%, 0.05 to 0.25%, 0.075 to 0.25%, 0.10 to 0.25%, 0.15 to 0.25%, 0.20 to 0.25%, 0 0.04 to 0.20%, 0.05 to 0.2%, 0.05 to 0.15%, 0.075 to 0.10%, 0.09 to 0.10%, and in particular about 0.05% .

The Juveleven™ product from Lipotec is a solution comprising 0.05% acetyl-hexapeptide-51 amide, as a percentage by weight relative to the total weight of the commercial product.

A quantity of acetyl-hexapeptide-51 amide ranging from 0.00025 to 0.0025% is therefore equivalent to a quantity of the Juveleven™ product ranging from 0.5 to 5%.

According to another particular embodiment, the present invention relates to a combination as defined above, in which the quantity of hyaluronic acid is comprised from 0.1 to 2%, preferably from 0.25 to 1%
as a percentage by weight relative to the total weight of the combination.

“From 0.1 to 2%” also means the following ranges: 0.2 to 2%, 0.3 to 2%, 0.4 to 2%, 0.5 to 2%, 0.6 to 2%, 0.8 to 2%, 1 to 2%, 1.2 to 2%, 1.4 to 2%, 1.6 to 2%, 1.8 to 2%, 0.1 to 1.8% , 0.1 to 1.6%, 0.1 to 1,4%, 0.1 to 1.2%, 0.1 to 1%, 0.1 to 0.8%, 0.1 to 0.6%, 0.1 to 0.4%, 0.1 to 0, 2%, 0.2-1.9%, 0.3-1.8%, 0.4-1.7%, 0.5-1.6%, 0.6-1.5%, 0, 7 to 1.4%, 0.8 to 1.3%, 0.9 to 1.2%, and in particular from 0.25 to 1%.

According to another particular embodiment, the present invention relates to a combination as defined above, in which the quantity of hyaluronic acid is comprised from 1 to 25%, preferably from 10 to 25%.

According to another particular embodiment, the present invention relates to a combination as defined above, in which the quantity of the tulip extract is greater than 0% and less than or equal to 0.05%, in particular comprised of 0.001 to 0.05%, in particular from 0.01 to 0.04%, as a percentage by weight relative to the total weight of the combination, and/or
in which the quantity of acetyl-tetrapeptide-2 is between 0.025 and 0.25% , in particular from 0.05 to 0.15%, preferably approximately 0.05%, as a percentage by weight relative to the total weight of the combination, and/or
in which the quantity of acetyl-hexapeptide-51 amide ranges from 0.025 to 0.25%, in particular from 0.05 to 0.2%, preferably approximately 0.1%, as a percentage by weight relative to the total weight of the combination, and/or
in which the amount of hyaluronic acid is between 0.1 and 2%, preferably 0.25 and 1%, as a percentage by weight relative to the total weight of the combination.

According to another particular embodiment, the present invention relates to a combination as defined above, comprising or consisting of:
a purple tulip extract, at a rate of 0.1% to 5%, as a percentage by weight relative to the total weight of the combination, and
acetyl-tetrapeptide-2, at a rate of 0.005 to 0.05%, as a percentage by weight relative to the total weight of the combination, and
acetyl-hexapeptide-51 amide, at a rate of 0.005 to 0.05%, as a percentage by weight relative to the total weight of the combination.

According to another particular embodiment, the present invention relates to a combination as defined above, comprising or consisting of:
a purple tulip extract, at a rate of 0.1 to 5%, as a percentage by weight relative to the total weight of the combination, and
acetyl-tetrapeptide-2, at a rate of 0.005 to 0.05%, as a percentage by weight relative to the total weight of the combination, and
acetyl-hexapeptide-51 amide, at a rate of 0.005 to 0.05%, as a percentage by weight relative to the total weight of the combination,
hyaluronic acid, at a rate of 1 to 25%, as a percentage by weight relative to the total weight of the combination.

According to another particular embodiment, the present invention relates to a combination as defined above, comprising or consisting of:
a purple tulip extract, at a rate of 0.001 to 0.05%, as a percentage by weight relative to the total weight of the combination, and
acetyl-tetrapeptide-2, at a rate of 0.025 to 0.25%, as a percentage by weight relative to the total weight of the combination, and
acetyl-hexapeptide-51 amide, at a rate of 0.025 to 0.25%, as a percentage by weight relative to the total weight of the combination.

According to another particular embodiment, the present invention relates to a combination as defined above, comprising or consisting of:
a purple tulip extract, at a rate of 0.001 to 0.05%, as a percentage by weight relative to the total weight of the combination, and
acetyl-tetrapeptide-2, at a rate of 0.025 to 0.25%, as a percentage by weight relative to the total weight of the combination, and
acetyl-hexapeptide-51 amide, at a rate of 0.025 to 0.25%, as a percentage by weight relative to the total weight of the combination,
hyaluronic acid, at a rate of 0.1 to 2%, as a percentage by weight relative to the total weight of the combination.

A second object of the present invention is the use of a combination as defined above, as a cosmetic composition.

By “cosmetic composition” is meant any composition for cosmetic purposes, in particular a composition that can be brought into contact with the surface parts of the human body, the skin, in particular the epidermis.

Within the meaning of the present invention, the term “cosmetic composition” means a non-pharmaceutical composition, that is to say which does not involve therapeutic treatment, that is to say intended to be used on an area of healthy skin.

The term “healthy skin” is understood to mean an area of skin to which the combination or composition according to the invention is applied, said to be “non-pathological” by a dermatologist, that is to say not showing any infection, disease, or sores or injuries and/or other dermatitis.

A third object of the present invention is a cosmetic composition, comprising a combination as defined above, in a cosmetically acceptable medium.

By “a cosmetically acceptable medium”, is meant within the meaning of the invention a medium compatible with use in cosmetics.

According to another particular embodiment, the present invention relates to a cosmetic composition as defined above comprising the following ingredients:
a tulip extract from the Tulipa Gesneriana family, in particular a purple tulip extract, and
acetyl-tetrapeptide-2, and
acetyl-hexapeptide-51 amide, and optionally
hyaluronic acid whose molecular mass is between 20 and 50 kDa.

According to another particular embodiment, the present invention relates to a cosmetic composition as defined above, in which the quantity of the extract of tulip, in particular of purple tulip, comprised from 0.01 to 0.05%, in particular from 0.02 to 0.04%, in percentage by weight relative to the total weight of the composition.

According to another particular embodiment, the present invention relates to a cosmetic composition as defined above, in which the amount of acetyl-tetrapeptide-2 is between 0.00025 and 0.0025%, in particular 0.0005 to 0.001 in percentage by weight relative to the total weight of the composition.

According to another particular embodiment, the present invention relates to a cosmetic composition as defined above, in which the quantity of acetyl-hexapeptide-51 amide is between 0.00025 and 0.0025%, in particular from 0. 0005 to 0.001 in percentage by weight relative to the total weight of the composition.

According to one particular embodiment, the present invention relates to a cosmetic composition as defined above, said composition comprising at least one other active principle.

said other active ingredient is chosen in particular from: moisturizing agents, chemical filters, in particular UV A filters, mineral filters, in particular titanium dioxide, thermal waters, in particular Treignac® water.

Among the “moisturizing agents”, we can cite, by way of example, sodium lactate, polyols, in particular glycerin, propylene glycol, butylene glycol, pentylene glycol, hexylene glycol, dipropylene glycol, diethylene glycol mannitol and amino acids, caprylic/capric triglyceride, or a mixture of these agents.

Among the “chemical filters” we can cite, by way of example:
anthranilates, such as menthyl anthranilate,
benzophenones, such as benzophenone-2 (oxybenzone), benzophenone-4 (Uvinul®MS40),
benzylidene-camphors, such as 4-methylbenzylidene camphor (Eusolex®6300),
benzimidazoles, such as benzimidazilate (Neo Heliopan®AP), or phenylbenzimidazole sulphonic acid (Eusolex®232),
benzotriazoles, such as methylene bis-benzotriazolyltetramethylbutylphenol (Tinosorb®M),
cinnamates, such as ethocrylene (Uvinul®N35), octylmethoxycinnamate (Parsol®MCX), or octocrylene (Uvinul®539),
dibenzoylmethanes, such as butyl methoxydibenzoylmethane (Parsol®1789),
imidazolines, such as ethylhexyl dimethoxybenzylidene dioxoimidazoline,
PABAs, such as diethylhexylbutamido-triazone (Uvasorb®HEB), ethylhexyltriazone (Uvinul®TI 50), or ethyl PABA (benzocaine),
salicylates, such as dipropylene glycol salicylate, ethylhexyl salicylate, homosalate, or TEA salicylate,
triazines, such as anisotriazine (Tinosorb®), or a mixture of these filters.

Among the “mineral filters”, we can cite, by way of example, zinc oxide (ZnO) or titanium dioxide (T1O2), or a mixture of these filters.

The composition of the present invention may also contain other adjuvants and excipients usual in the cosmetics fields, such as for example cosmetic oils, in particular oils containing a-linolenic acid, antioxidants, preservatives, emulsifiers, hydrophilic or lipophilic gelling agents, hydrophilic or lipophilic active agents.

The term “cosmetic oils” refers to oils compatible with cosmetic use.

The “cosmetic oils” according to the present invention may be, or may contain, by way of example:
triglyceride-based vegetable oils such as sunflower oil, sesame oil, rapeseed oil, sweet almond oil, calophyllum oil, palm oil, avocado oil, jojoba oil, olive oil, castor oil or cereal germ oils such as wheat germ oil, apricot oil,
fatty acids such as a-linolenic acid, or containing a-linolenic acid,
esters of fatty acids and alcohols or polyols such as isopropyl, butyl or cetyl myristates, isopropyl, butyl or ethyl-2-hexyl palmitates, isopropyl stearates , butyl, octyl, hexadecyl or isocetyl, decyl oleate, hexyl laurate, esters derived from lanolic acid such as isopropyl or isocetyl lanolates, isononyl isononanoate, diisopropyl adipate, propylene glycol dicaprylate, glycol or glycerol octanoates and decanoates as well as cetyl ricinoleate, or a mixture of these oils.

As “antioxidants”, we can cite, for example, tocopherol (vitamin E) or ascorbic acid (vitamin C).

As “preservatives”, we can cite, for example, benzalkonium chloride, phenoxyethanol, or sorbic acid.

As “emulsifiers”, we can cite, for example, polyol fatty acid esters, for example glyceryl stearate, PEG-40 stearate, sorbitan tristearate, polyoxyethylene sorbitan stearates (Tween®-60 or Tween®-20), ceteareth-20 (ethoxylated), cetyl alcohol.

As “hydrophilic gelling agent”, we can cite, for example, carboxyvinyl polymers, acrylic copolymers, polysaccharides, natural gums, such as xanthan gum, clays, Sepigel™305.

As a “lipophilic gelling agent” we can cite hydrophobic silica.

Said adjuvants and other active principles can be present in the composition in amounts conventionally used in cosmetics, in particular from 0.01 to 20% in percentage by weight relative to the total weight of the composition.

“From 0.01 to 20%” also means the following ranges: 1 to 20%, 2 to 20%, 5 to 20%, 10 to 20%, 15 to 20%, 0.01 to 15%, 0.01 to 10%, 0.01 to 5%, 0.01 to 2%, 0.01 to 1%, 1 to 15%, 2 to 10%, and 3 to 5%.

The present invention also relates to a cosmetic composition, comprising a combination as defined above, in a cosmetically acceptable medium, said composition optionally comprising at least one other active principle, in particular chosen from: moisturizing agents, chemical filters, in particular UV A filters, mineral filters, in particular titanium dioxide, thermal waters, in particular Treignac® water.

According to another particular embodiment, the present invention relates to a cosmetic composition as defined above, said cosmetic composition being formulated for topical application, in particular topical application to the skin.

According to another particular embodiment, the present invention relates to a cosmetic composition as defined above, said composition being in the form of an emulsion, a cream, a gel, a dispersion, a serum, foam, body milk or anhydrous balm.

An emulsion according to the present invention can be an oil-in-water emulsion, or a water-in-oil emulsion.

The fats and emulsifiers present in the emulsions according to the present invention are those usually used in cosmetics.

The fats that can be used are, for example, mineral or vegetable oils,

The emulsifiers can in particular be chosen from polyol fatty acid esters, for example glyceryl stearate, PEG-40 stearate, sorbitan tristearate, polyoxyethylene sorbitan stearates (Tween®-60 or Tween®-20), ceteareth-20 (ethoxylated).

According to another particular embodiment, the present invention relates to a cosmetic composition as defined above, said composition being in the form of a day cream.

According to another particular embodiment, the present invention relates to a cosmetic composition as defined above, said composition being in the form of a night cream.

According to another particular embodiment, the present invention relates to a cosmetic composition as defined above, said cosmetic composition being formulated for topical application, in particular topical application to the skin, said composition being in particular in the form of a emulsion, cream, gel, dispersion, serum, mousse, body milk or anhydrous balm, Especially in the form of a day cream, or a night cream.

A fourth object of the present invention is the use of a combination as defined above, as a pharmaceutical, in particular dermatological composition.

By “pharmaceutical composition” is meant any composition for pharmaceutical purposes, in particular a dermatological composition which can be brought into contact with the superficial parts of the human body, the skin, in particular the epidermis.

A fifth object of the present invention is a dermatological composition, comprising, as active substance, a combination as defined above, with a pharmaceutically acceptable excipient.

By “pharmaceutically acceptable excipient” is meant within the meaning of the invention any compound making it possible to facilitate the shaping of the composition and not modifying the nature of the biological activity of the active principle, with a pharmaceutical use. A pharmaceutically acceptable excipient can be, by way of example, a solvent, plasticizer, lubricant, dispersing medium, agents delaying absorption, flow agent.

According to another particular embodiment, the subject of the present invention is a dermatological composition as defined above, said dermatological composition being formulated for topical application, in particular topical application to the skin.

According to another particular embodiment, the subject of the present invention is a dermatological composition as defined above, said composition being in the form of an emulsion, a cream, a gel, a dispersion, serum, mousse, body milk or anhydrous balm.

The present invention also relates to a dermatological composition, comprising, as active substance, a combination as defined above, with a pharmaceutically acceptable excipient, said dermatological composition being in particular formulated for topical application, in particular topical application of the skin, said composition being in particular in the form of an emulsion, a cream, a gel, a dispersion, a serum, a mousse, a body milk or an anhydrous balm.

A sixth object of the present invention is the use of a combination as defined above, in the cosmetic treatment, or the cosmetic prevention of skin ageing.

Another object of the present invention is the use of a combination as defined above, in the non-therapeutic cosmetic treatment, or the non-therapeutic cosmetic prevention of skin ageing.

Within the meaning of the present invention, the term “cosmetic treatment” is understood to mean a non-therapeutic treatment, that is to say intended for any zone of healthy skin.

A seventh object of the present invention is the use of a cosmetic composition as defined above, in the cosmetic treatment or the cosmetic prevention of skin aging.

An eighth object of the present invention is a combination as defined above, for its use in the prevention or treatment of pathologies linked to oxidative stress.

A ninth object of the present invention is a dermatological composition as defined above, for its use in the prevention or treatment of pathologies linked to oxidative stress.

The inventors have unexpectedly found that the combination of ingredients of the cosmetic composition or of the combination according to the present invention, namely an extract of tulip of the species of Tulipa Gesneriana, in particular an extract of purple tulip, acetyl-tetrapeptide-2, acetyl-hexapeptide-51 amide, and optionally hyaluronic acid, can protect, prevent or treat all forms of aged skin.

It contributes to the restoration of the biomechanical properties of the skin, such as firmness, elasticity, and leads to a synergistic effect in the prevention or treatment of pathologies linked to oxidative stress.

The inventors have developed an oil-in-water emulsion cream with the proportions indicated in Table 1.

Said composition includes purple tulip extract (Dumaflorine®), acetyl-tetrapeptide-2 (Uplevity™), acetyl-hexapeptide-51 amide (Juveleven™) and hyaluronic acid.

The composition of Table 1 can be used both as a cosmetic composition or a dermatological composition.

TABLE 1
Oil-in-water emulsion cream composition

| Phase | Ingredient | m/m % |
|---|---|---|
| A | Solvent | QSP 100 |
| | moisturizing agent | 3 |
| | cosmetic oil 1 | 12 |
| | cosmetic oil 2 | 2 |
| | emulsifier | 2 |
| | Dumaflorine ® | 0.2 |
| | Juveleven ™ | 2 |
| | Uplevity ™ | 1 |
| | Hyaluronic acid | 1 |
| B | Oil 1 | 8 |
| | Oil 2 | 1 |
| | Oil 3 | 1 |
| C | preservative | 0.1 |

Another composition developed by the inventors is a composition with the proportions indicated in Table 2.

Said composition includes purple tulip extract (Dumaflorine®), acetyl-tetrapeptide-2 (Uplevity™), and acetyl-hexapeptide-51 amide (Juveleven™)

It is a cream-gel composition that can be used both as a cosmetic composition or a dermatological composition.

TABLE 2
gel cream composition

| Phase | Ingredient | m/m % |
|---|---|---|
| A | cosmetic oil | 6 |
| | cosmetic oil | 2 |
| | Antiseptic agent | 3 |
| | gelling agent | 3 |
| B | Solvent | 82.6 |
| | Dumaflorine ® | 0.4 |
| | Juveleven ™ | 2 |
| | Uplevity ™ | 1 |

Another object of the present invention is a cosmetic treatment method or cosmetic prevention of skin aging, comprising the application to the skin of a composition as defined above. Another object of the present invention is a non-therapeutic cosmetic treatment method or non-therapeutic cosmetic prevention of skin aging, comprising the application to the skin of a composition as defined above.

Within the meaning of the present invention, the term “cosmetic treatment method” means a method which does not require therapeutic treatment, that is to say a treatment method intended for any zone of healthy skin.

According to a particular embodiment, the present invention relates to a method as defined above, in which the said composition is applied from 1 to 3 times per day, in particular once in the morning and once in the evening to cleansed skin.

The following Figures and examples illustrate the invention, without limiting its scope.

### FIGURES

FIG. 1 represents an explanation of the analysis of the images according to example 10.

FIG. 1A represents an image of an immunostaining, in which a corresponds to the labeled structure.

FIG. 1B shows label detection, where b represents the label, selected by pixel thresholding.

FIG. 1C represents the determination of the region of interest, by manual tracing, in the case of the epidermis c.

FIG. 1D represents the combination of FIGS. 1B and 1C, in which d represents the labeling in the epidermis.

FIG. 2 represents the evaluation of hyaluronic acid in the epidermis (EpiD) and the papillary dermis (DP) of example 10B.
TF=very weak, F=weak, M=moderate, AN=fairly clear, N=clear, TN=very clear, Fo=strong.

FIG. 3 represents the evaluation of elastin in the papillary dermis of example 10C.
TF=very weak, F=weak, M=moderate, AN=fairly clear, N=clear, TN=very clear, Fo=strong.

FIG. 4 represents the evaluation of collagen I in the papillary dermis of example 10D.
TF=very weak, F=weak, M=moderate, AN=fairly clear, N=clear, TN=very clear, Fo=strong.

### EXAMPLES

### Definition of Trademarks

| Trademarks | INCI | Name |
|---|---|---|
| Dumaflorine ® | Maltodextrin, Tulipa gesneriana | Maltodextrin, Tulipa gesneriana |
| | flower extract | flower extract |
| Juveleven ™ | Butylene glycol/Aqua/acetyl | Butylene glycol/Aqua/acetyl |
| | hexapeptide-51 amide | hexapeptide-51 amide |
| Uplevity ™ | Aqua/Acetyl Tetrapeptide- | Water/Acetyl Tetrapeptide- |
| | 2/caprylyl glycol | 2/caprylyl glycol |
| Dow Corning ® | Cyclopentasiloxane/Dimethicone | Cyclopentasiloxane/Dimethicone |
| 9040 | Crosspolymer | Crosspolymer |
| Regu ®-Seb | Serenoa Serrulata Fruit | saw palmetto extract, sesame |
| | Extract/Sesamum Indicum | seed oil, argan oil, phytosterol, |
| | (Sesame) Seed Oil/Argania | tocopherol |
| | Spinosa Kernel Oil/Beta- | |
| | Sitosterol/Tocopherol | |
| Sepigel 305 ™ | Polyacrylamide/C13-14 | Polyacrylamide/C13-14 |
| | Isoparaffin/Laureth-7 | Isoparaffine/Laureth-7 |
| Easynov ™ | Octyldodecanol/Octyldodecyl | Octyldodecanol/Octyldodécyl |
| | Xyloside/PEG-30 | Xyloside/PEG-30 |
| | Dipolyhydroxystearate | Dipolyhydroxystearate |
| Uvinul ® MS40 | Benzophenone-4 | Sulisobenzone |
| Eusolex ® 6300 | Methylbenzylidene Camphor | methylbenzylidene camphor |
| Eusolex ® 232 | Phenylbenzimidazole Sulfonic | Phenylbenzimidazole Sulfonic |
| | Acid | Acid |
| Tinosorb ® M | Methylene Bis-Benzotriazolyl | Methylene Bis-Benzotriazolyl |
| | Tetramethylbutylphenol (and) | Tetramethylbutylphenol (and) |
| | Aqua (and) Decyl Glucoside | Aqua (and) Decyl Glucoside |
| | (and) Propylene Glycol (and) | (and) Propylene Glycol (and) |
| | Xanthan Gum | Xanthan Gum |
| Uvinul ® N35 | Etocrylene | Etocrylene |
| Uvinul ® 539 | Octocrylene | Octocrylene |
| Parsol ® 1789 | Butyl Methoxydibenzoylmethane | Avobenzone |
| Uvasorb ® HEB | Diethylhexyl Butamido Triazone | Iscotrizinol |
| Uvinul ® T150 | Ethyl hexyl triazone | Ethylhexyltriazone |
| Tinosorb ® | Bis-Ethylhexyloxyphenol | Bis-ethylhexyloxyphenol |
| | Methoxyphenyl Triazine | Methoxyphenyl Triazine |
| Tween ®-60 | Polysorbate 60 | Polysorbate 60 |
| Tween ®-20 | Polysorbate 20 | Polysorbate 20 |
| Sepigel ™ 305 | Polyacrylamide & C13-14 | Polyacrylamide, C13-14 |
| | Isoparaffin & Laureth-7 | Isoparaffine, Laureth-7 |
| Primalhyal ™ 50 | Hydrolyzed Hyaluronic Acid | Hydrolyzed Hyaluronic Acid |
| Sepimax Zen ™ | Polyacrylate Crosspolymer-6 | Polyacrylate Crosspolymer-6 |
| Euxyl ® K900 | Benzyl Alcohol, | Benzyl Alcohol, |
| | Ethylhexylglycerin, Tocopherol | Ethylhexylglycerin, Tocopherol |

Example 1: Evaluation of the ex vivo anti-aging activity of a cream and a serum

A cream and a serum comprising Dumaflorine®, Uplevity™ and Juveleven™ are prepared and tested ex vivo on explanted human skin.

12 Explants of human skin are prepared from a plasty taken from a person over the age of 40. The explants are left to survive at 37° C. in an atmosphere enriched with 5% CO₂.

The products are applied topically at a rate of 2mg/cm² and the treatment is renewed every two days.

The application of the products was carried out on three explants per product tested.

Six control explants were also prepared, including three plasticized controls (frozen at D0 ) and three untreated controls, undergoing the same survival as the treated explants.

The controls receive no treatment, or are treated with the excipient.

At D0, the three explants of the plasty control are removed and frozen at −80° C.

At D10, three explants from each batch are removed and frozen at −80° C.

The anti-aging activity is evaluated on the 12 explants by:
A viability check after staining
Staining of elastin
Immunolabelling of collagen I
Immunolabeling of hyaluronic acid

### Histological treatments

The frozen samples are cut at 7 μm for immunostaining.

Viability check. Paraffin sections are stained. A viability review is performed. Explants treated with cream or serum show, after 10 days, good viability of dermal and epidermal structures

### Immunolabelling of Collagen I

Collagen I is highlighted on sections thanks to a specific antibody.

The explants treated with the cream or the serum show, after 10 days, an increase in the expression of collagen I, compared to the controls.

### Immunolabelling of HABP

HABP is highlighted on sections thanks to a specific antibody.

After 10 days, an increase in the fluorescence is observed at the level of the epidermis of the skin samples treated with the cream or the serum, compared with the controls.

### Elastin Staining

Elastin is highlighted on sections thanks to a specific stain.

The explants treated with the cream or the serum show, after 10 days, an increase in the expression of elastin, compared to the controls.

Example 2: Study of the in vivo anti-aging activity of a cream and a serum

A cream and a serum comprising Dumaflorine®, Uplevity™ and Juveleven™ are prepared and tested in vivo on a panel of 20 volunteers, half-face.

Different assessments are carried out:
Evaluation of wrinkles, by projection of fringes
Evaluation of mechanical properties by cutometer
Taking pictures of the face
A self-assessment questionnaire targeting the effect felt, the effectiveness and the cosmetic quality of the product.

### Selection of Volunteers

The volunteers are recruited from the general population: subjects aged 45 to 65 with, in particular, wrinkles and fine lines around the crow's feet.

Course of the study

Day 0 (D0)
Discounts of the product to use and the satisfaction questionnaire
Performing biometrological measurements on each half-face:
   projection of fringes
   Cutometer
   Pictures

Day 1 to Day 28 (D1 to D28)
application of the product daily on the half-face.

Day 28 (D28)
Verification of the running of the study
Performing biometrological measurements on each half-face:
   projection of fringes
   Cutometer
   Pictures

Day 28 to Day 56 (D28 to D56)
application of the product daily on the half-face.

Day 56 (D56)
Verification of the running of the study
Performing biometrological measurements on each half-face:
   projection of fringes
   Cutometer
   Pictures
Return of the questionnaire and return of the products

### Evaluation of Mechanical Properties

The evaluation of the mechanical properties makes it possible to study the functional level of the tissue structures responsible for the firmness of the skin:

Elastic structures (elastic fibers, curvature of connective bundles, folding of the stratum comeum)

Structures with viscous behavior (interstitial fluids, internal adhesions).

The study is carried out by the cutometer whose measurements are based on the principle of the suction method by creating a negative pressure (between 20 and 500 mbar) carried out in a cylindric “chamber”. Successive cycles of suction-release programmed in the software make it possible, after each measurement, to record the graph of the deformation as a function of time and then to obtain by a cursor system all the desired extension parameters.

Schematically, the resistance of the skin to being deformed by negative pressure during the suction phase measures skin tone, while its ability to return to its original position during the relaxation phase measures skin elasticity.

Conventionally, 3 parameters measuring ratios are taken into account:
RO, representing the firmness of the skin. It is the passive behavior of the skin in relation to the force applied.
R6 during the suction phase measuring the visco-elastic properties of the skin (if skin elasticity improves, this R6 ratio will decrease)
R7 during the relaxation phase measuring the pure biological elasticity of the skin (if skin elasticity improves, this R7 ratio will increase).

### Taking Pictures

Facial photographs are taken and wrinkles are analyzed.

The present in vivo study demonstrated the effectiveness of the cream and serum tested.

Example 3: Study of the effects of a combination comprising Dumaflorine®, Uplevity™ and Juveleven™

A combination comprising Dumaflorine®, Uplevity™ and Juveleven™ was used and tested in vitro. Protection of the DNA of cells against damage caused by benzo[a]pyrene, a persistent pollutant contained in cigarette smoke or automobile exhaust, has been observed.

The combination has been used and tested in vivo. Significant repair of UV DNA damage was observed.

Example 4: Study of the effects of a combination comprising Dumaflorine®, Uplevity™ and Juveleven™

A combination comprising Dumaflorine®, Uplevity™ and Juveleven™ has been used and tested in vivo on mature skin. An increase in firmness was observed, as well as an improvement in the integrity of the dermis (assessed by ballistometry and confocal microscopy).

Example 5: oil-in-water emulsion cream

TABLE 3
Oil-in-water emulsion cream composition

| Phase | Ingredient | INCI | m/m % |
|---|---|---|---|
| A | Water | Aqua | QSP 100 |
| | Glycerin | Glycerin | 3 |
| | Glyceryl | Glyceryl stearate | 12 |
| | stearate | | |
| | Cetyl alcohol | Cetyl alcohol | 2 |
| | Ceteareth-20 | Ceteareth-20 | 2 |
| | Dumaflorine ® | Maltodextrin, Tulipa gesneriana | 0.2 |
| | | flower extract | |
| | Juveleven ™ | Butylene glycol/Aqua/acetyl | 2 |
| | | hexapeptide-51 amide | |
| | Uplevity ™ | Aqua/Acetyl Tetrapeptide-2/ | 1 |
| | | caprylyl glycol | |
| B | Caprylic/capric | Caprylic/capric triglyceride | 8 |
| | triglyceride | | |
| | Argan oil | Argania Spinosa Oil | 1 |
| | sweet almond | Prunus dulcis oil | 1 |
| | oil | | |
| C | Phenoxyethanol | Phenoxyethanol | 0.1 |

Example 6: gel cream

TABLE 4
gel cream composition

| Phase | Ingredient | INCI | m/m % |
|---|---|---|---|
| A | Dow | Cyclopentasiloxane/Dimethicone | 6 |
| | Corning ® 9040 | Crosspolymer | |
| | apricot oil | Prunus Armeniaca kernel oil | 2 |
| | Regu ®-Seb | Serenoa Serrulata Fruit Extract/ | 3 |
| | | Sesamum Indicum (Sesame) Seed | |
| | | Oil/Argania Spinosa Kernel Oil/ | |
| | | Beta-Sitosterol/Tocopherol | |
| | Sepigel 305 ™ | Polyacrylamide/C13-14 Isoparaffin/ | 3 |
| | | Laureth-7 | |
| B | Water | Aqua | 82.6 |
| | Dumaflorine ® | Maltodextrin, Tulipa gesneriana | 0.4 |
| | | flower extract | |
| | Juveleven ™ | Butylene glycol/Aqua/acetyl | 2 |
| | | hexapeptide-51 amide | |
| | Uplevity ™ | Aqua/Acetyl Tetrapeptide-2/ | 1 |
| | | caprylyl glycol | |

Example 7: Water-in-oil cosmetic milk

TABLE 5
Water-in-oil cosmetic milk composition

| Phase | Ingredient | INCI | m/m % |
|---|---|---|---|
| A | Water | Aqua | 64.5 |
| | Glycerin | Glycerin | 3 |
| | Xanthan gum | Xanthan gum | 1 |
| | Glyceryl | Glyceryl stearate | 12 |
| | stearate | | |
| | shea butter | Butyrospermum parkii butter | 2 |
| | Easynov ™ | Octyldodecanol/Octyldodecyl | 4 |
| | | Xyloside/PEG-30 | |
| | | Dipolyhydroxystearate | |
| | Dumaflorine ® | Maltodextrin, Tulipa gesneriana | 0.4 |
| | | flower extract | |
| | Juveleven ™ | Butylene glycol/Aqua/acetyl | 2 |
| | | hexapeptide-51 amide | |
| | Uplevity ™ | Aqua/Acetyl Tetrapeptide-2/ | 1 |
| | | caprylyl glycol | |
| B | Caprylic/capric | Caprylic/capric triglyceride | 4 |
| | triglyceride | | |
| | Isopropyl | Isopropyl myristate | 4 |
| | myristate | | |
| | Argan oil | Argania Spinosa Oil | 1 |
| | sweet almond | Prunus dulcis oil | 1 |
| | oil | | |
| C | Phenoxyethanol | Phenoxyethanol | 0.1 |

Example 8: oil-in-water emulsion cream comprising hyaluronic acid

TABLE 6
Oil-in-water emulsion cream composition

| Phase | Ingredient | INCI | m/m % |
|---|---|---|---|
| A | Water | Aqua | QSP 100 |
| | Glycerin | Glycerin | 3 |
| | Glyceryl | Glyceryl stearate | 12 |
| | stearate | | |
| | Cetyl alcohol | Cetyl alcohol | 2 |
| | Ceteareth-20 | Ceteareth-20 | 2 |
| | Dumaflorine ® | Maltodextrin, Tulipa gesneriana | 0.2 |
| | | flower extract | |
| | Juveleven ™ | Butylene glycol/Aqua/acetyl | 2 |
| | | hexapeptide-51 amide | |
| | Uplevity ™ | Aqua/Acetyl Tetrapeptide-2/ | 1 |
| | | caprylyl glycol | |
| | Hyaluronic acid | Hyaluronic acid | 1 |
| B | Caprylic/capric | Caprylic/capric triglyceride | 8 |
| | triglyceride | | |
| | Argan oil | Argania Spinosa Oil | 1 |
| | sweet almond | Prunus dulcis oil | 1 |
| | oil | | |
| C | Phenoxyethanol | Phenoxyethanol | 0.1 |

Example 9: Water-in-oil cosmetic milk comprising hyaluronic acid

TABLE 7
Water-in-oil cosmetic milk composition

| Phase | Ingredient | INCI | m/m % |
|---|---|---|---|
| A | Water | Aqua | 64.5 |
| | Glycerin | Glycerin | 3 |
| | Gomme Xanthane | Xanthan gum | 1 |
| | Glyceryl | Glyceryl stearate | 12 |
| | stearate | | |
| | shea butter | Butyrospermum parkii butter | 2 |
| | Easynov ™ | Octyldodecanol/Octyldodecyl | 4 |
| | | Xyloside/PEG-30 | |
| | | Dipolyhydroxystearate | |
| | Dumaflorine ® | Maltodextrin, Tulipa gesneriana | 0.4 |
| | | flower extract | |
| | Juveleven ™ | Butylene glycol/Aqua/acetyl | 1 |
| | | hexapeptide-51 amide | |
| | Uplevity ™ | Aqua/Acetyl Tetrapeptide-2/ | 1 |
| | | caprylyl glycol | |
| | Hyaluronic acid | Hyaluronic acid | 1 |
| B | Caprylic/capric | Caprylic/capric triglyceride | 4 |
| | triglyceride | | |
| | Isopropyl | Isopropyl myristate | 4 |
| | myristate | | |
| | Argan oil | Argania Spinosa Oil | 1 |
| | sweet almond | Prunus dulcis oil | 1 |
| | oil | | |
| C | Phenoxyethanol | Phenoxyethanol | 0.1 |

Example 10: Evaluation of the anti-aging action

2 compositions, a cream according to example 5 (P1) and a serum (P2), were prepared and tested on ex vivo human skin explants.

| Phase | Ingredient | INCI | m/m % |
|---|---|---|---|
| A | Water | Aqua | 89.8 |
| | isopentyldiol | isopentyldiol | 5.0 |
| | Dumaflorine ® | Maltodextrin, Tulipa gesneriana | 0.3 |
| | | flower extract | |
| | Primalhyal ™ 50 | Hydrolyzed Hyaluronic Acid | 0.1 |
| B | Sepimax Zen ™ | Polyacrylate Crosspolymer-6 | 0.3 |
| C | Euxyl ® K900 | Benzyl Alcohol, Ethylhexylglycerin, | 1.5 |
| | | Tocopherol | |
| | Juveleven ™ | Butylene glycol/Aqua/acetyl | 2.0 |
| | | hexapeptide-51 amide | |
| | Uplevity ™ | Aqua/Acetyl Tetrapeptide-2/ | 1.0 |
| | | caprylyl glycol | |

### Preparation of Explants

12 explants 12±1 mm in diameter were prepared from a plasty of a 60-year-old woman (reference P2258-AB60).

The explants were left to survive in BEM medium (BIO-EC's Expiants Medium) at 37° C. in a humid atmosphere, enriched with 5% CO2.

### Distribution of Explants

The explants were divided into 4 batches as indicated in Table 9:

| Batch | Designation | Treatment | Explants number | Sample |
|---|---|---|---|---|
| T0 | Plasty control | none | 3 | D0 |
| T | Non treated | none | 3 | D10 |
| | control | | | |
| P1 | Composition P1 | P1 | 3 | D10 |
| P2 | Composition P2 | P2 | 3 | D10 |

### Application of products

At D0 , D2, D4, D7 and D9, the P1 and P2 products were applied topically, at a rate of 2 μl per explant (2 mg/cm²) and spread using a spatula.

The explants of batches T did not receive any treatment, except renewal of the medium.

Half of the medium was renewed (1 ml per well) at D2, D4, D7 and D9.

### Specimens

At D0 , the 3 explants of batch T0 were removed and cut in half.

One part was fixed in buffered formalin solution, and the other was frozen at −80° C.

At D10, 3 explants from each batch were collected and treated in the same way as at D0 .

### Histological Treatments

After 24 hours in buffered formalin, the samples were dehydrated and impregnated with paraffin using a Leica PEARL dehydration machine. They were blended using a Leica EG 1160 embedding station.

5 μm sections were made using a Minot type microtome, Leica RM 2125 and mounted on Superfrost® histological glass slides.

7 μm sections were made from the frozen samples using a Leica CM 3050 cryostat and mounted on Superfrost®Plus histological glass slides.

The microscopic observations were carried out by optical microscopy, using a Leica type DMLB or Olympus BX43 or BX63 microscope.

The shots were taken with an Olympus DP72 or DP74 camera and cellSens software.

### Image Analysis

Image analyzes were performed on all photos from each batch using cellSens Olympus software.

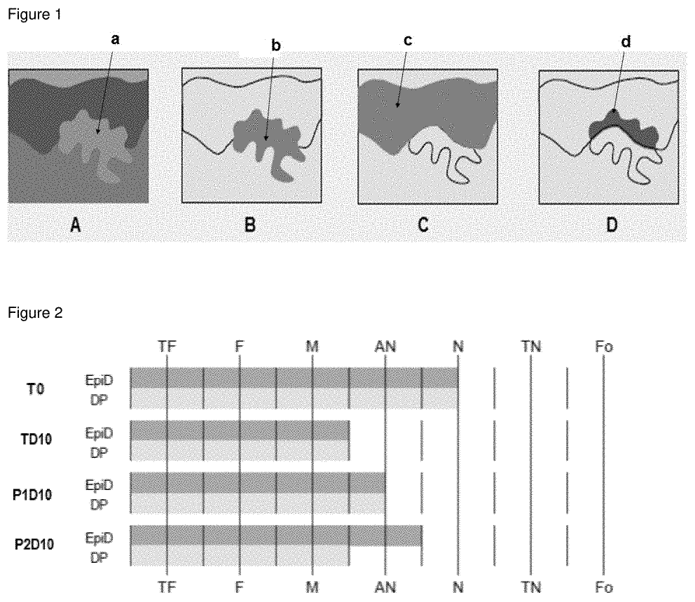

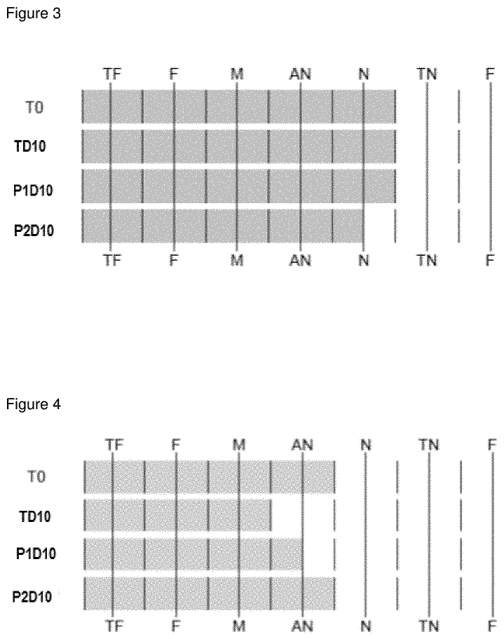

For each batch of explants, the percentage of surface of the region of interest covered by the marking (percentage of marked surface) is determined by an image analysis according to FIG. 1.

The percentage of marked surface (Surf%) for each treatment is compared to the condition T=>P vs T.

Example 10A: viability check after staining with Masson's trichrome

The cellular viability of the epidermal and dermal structures was observed on paraffin sections after staining with Masson's trichrome variant of Goldner.

It was assessed by microscopic examination.

Cell viability for all batches is shown in Table 10:

TABLE 10
cell reliability results

Cell viability

| Batch | Epiderm | Derm |
|---|---|---|
| T0 | B | B |
| TD10 | AB | B |
| P1D10 | AB | B |
| P2D10 | AB | B |

morphology caption: B = good, AB = Fair

Example 10B: immunostaining of hyaluronic acid by HABP

Labeling of hyaluronic acid was carried out on paraffin sections with a biotinylated HABP (hyluronic acid binding protein) (amsbio, ref. AMS.HKD.BC41) diluted 1/800th in PBS-BSA 0.3%, for 1 hour at room temperature, then amplified with a biotin/streptavidin system and revealed in VIP, a purple peroxidase substrate (Vector laboratories, SK-4600).

Immunolabelling was performed manually, evaluated by microscopic observation and semi-quantified by image analysis.

The evaluation of hyaluronic acid (HABP) in the epidermis (EpiD) and the papillary dermis (DP) of all batches is in FIG. 2:

### In Living Epidermis

On the control batch T0 , the labeling of hyaluronic acid is clear in the living epidermis.

On the control batch TD10, the synthesis of hyaluronic acid is moderate to fairly clear in the living epidermis.

Effect of product application on hyaluronic acid synthesis, compared to batch TD10:
The P1 composition induces a slight increase
The P2 composition induces a moderate increase.

### In the Papillary Dermis

On the control group T0 , the labeling of hyaluronic acid is clear in the papillary dermis.

On the TD10 control batch, hyaluronic acid synthesis is moderate to fairly clear in the papillary dermis.

Effect of products on hyaluronic acid synthesis, compared to batch TD10:
The P1 composition induces a slight increase
The P2 composition does not induce any modification.

The percentage of surface occupied by hyaluronic acid (HABP) in the living epidermis of all batches is shown in Table 11:

TABLE 11

| | T0 | TD10 | P1D10 | P2D10 |
|---|---|---|---|---|
| Mean | 36.3 | 18.1 | 24.5 | 30.5 |
| Standard Deviation | 7.4 | 6.2 | 7.0 | 6.8 |

The percentage of surface occupied by hyaluronic acid (HABP) in the papillary dermis of all batches is shown in Table 12:

TABLE 12

| | T0 | TD10 | P1D10 | P2D10 |
|---|---|---|---|---|
| Mean | 74.3 | 44.1 | 57.1 | 41.9 |
| Standard Deviation | 6.3 | 7.1 | 12.1 | 11.7 |

Example 10C: immunostaining of elastin

Elastin was labeled on paraffin sections with a polyclonal anti-elastin antibody (Novotec, ref. 25011), diluted to 1/100th in PBS-BSA 0.3%-Tween 20 at 0.05% for 1 hour at temperature ambient and revealed in AlexaFluor488 (Lifetechnologies, ref. Al 1008). The nuclei were counterstained with propidium iodide.

Immunolabeling was performed using an immunolabeling automaton (Dako, AutostainerPlus), evaluated by microscopic examination and semi-quantified by image analysis.

Elastin labeling in the papillary dermis of all batches is shown in FIG. 3.

The percentage of surface occupied by elastin in the papillary epidermis of all batches is shown in Table 13:

| | T0 | TD10 | P1D10 | P2D10 |
|---|---|---|---|---|
| Mean | 13.6 | 13.0 | 14.2 | 10.3 |
| Standard Deviation | 3.6 | 3.9 | 3.0 | 1.6 |

Example 10D: immunostaining of collagen I

Collagen I was labeled on frozen sections with an anti-collagen I polyclonal antibody (Monosan, ref. PS047), diluted to 1/100th in 0.05% PBS-BSA 0.3%-Tween 20 for 1 hour at room temperature and revealed in AlexaFluor488 (Lifetechnologies, ref. Al 1008).

The nuclei were counterstained with propidium iodide.

Immunolabeling was performed using an immunolabeling automaton (Dako, AutostainerPlus), evaluated by microscopic examination and semi-quantified by image analysis.

The evaluation of collagen I in the papillary dermis of all batches is shown in FIG. 4:

The percentage of surface occupied by elastin in collagen I of all batches is shown in the

Table 14:

TABLE 14

| | T0 | TD10 | P1D10 | P2D10 |
|---|---|---|---|---|
| Mean | 85.6 | 59.9 | 73.9 | 80.6 |
| Standard Deviation | 10.1 | 15.2 | 10.2 | 9.6 |

In conclusion, the “day cream” (P1) composition shows anti-aging activity, by inducing an increase in hyaluronic acid both in the living epidermis and in the papillary dermis as well as an increase in collagen I after 10 days of treatment.

The “serum composition” (P2) shows anti-aging activity, inducing an increase in hyaluronic acid in the living epidermis as well as an increase in collagen I after 10 days of treatment.

1-26. (canceled)

27. A combination comprising or consisting of the following three ingredients:
a tulip extract from the Tulipa Gesneriana family, and
acetyl-tetrapeptide-2, and
acetyl-hexapeptide-51 amide.

28. The combination according to claim 27, wherein the combination comprises hyaluronic acid whose molecular weight is between 20 and 50 kDa.

29. The combination according to claim 27, wherein the combination comprises maltodextrin.

30. The combination according to claim 27, comprising or consisting of the following four ingredients:
a tulip extract from the Tulipa Gesneriana family, and
acetyl-tetrapeptide-2, and
acetyl-hexapeptide-51 amide,
hyaluronic acid whose molecular mass is between 20 and 50 kDa, and optionally maltodextrin.

31. The combination according to claim 27, wherein the tulip extract comprises
20 to 70% of sugars, and
15 to 30% of phenolic compounds, of which at least 8% phenolic compounds are flavonoids,
in percentage by weight relative to the total weight of the dry extract,
said flavonoids comprise 50 to 90% of quercetin and 10 to 50% of kampferol,
in percentage by weight relative to the total weight of flavonoids.

32. The combination according to claim 27, wherein the quantity of the tulip extract is greater than 0% and less than or equal to 5%, as a percentage by weight relative to the total weight of the combination.

33. The combination according to claim 27, wherein the quantity of acetyl-tetrapeptide-2 is between 0.005 and 0.05%, in percentage by weight relative to the total weight of the combination.

32. The combination according to claim 27, wherein the quantity of acetyl-hexapeptide-51 amide is between 0.005 and 0.05%, as a percentage by weight relative to the total weight of the combination.

33. The combination according to claim 27, wherein the quantity of yaluronic acid is between 1 and 25%, as a percentage by weight relative to the total weight of the combination.

34. The combination according to claim 27, comprising or consisting of:
a tulip extract from the Tulipa Gesneriana family, at a rate of 0.1 to 5%, as a percentage by weight relative to the total weight of the combination, and
acetyl-tetrapeptide-2, at a rate of 0.005 to 0.05%, as a percentage by weight relative to the total weight of the combination, and
acetyl-hexapeptide-51 amide, at a rate of 0.005 to 0.05%, as a percentage by weight relative to the total weight of the combination.

35. The combination according to claim 27, comprising or consisting of:
a tulip extract from the Tulipa Gesneriana family, at a rate of 0.1 to 5%, as a percentage by weight relative to the total weight of the combination, and
acetyl-tetrapeptide-2, at a rate of 0.005 to 0.05%, as a percentage by weight relative to the total weight of the combination, and
acetyl-hexapeptide-51 amide, at a rate of 0.005 to 0.05%, as a percentage by weight relative to the total weight of the combination,
hyaluronic acid, at a rate of 1 to 25%, as a percentage by weight relative to the total weight of the combination.

36. A cosmetic composition, comprising a combination according to claim 27, in a cosmetically acceptable medium.

37. A dermatological composition, comprising, as an active substance, a combination according to claim 27, with a pharmaceutically acceptable excipient.

38. The dermatological composition according to claim 37, wherein said dermatological composition is formulated for topical application.

39. A method for treating or preventing pathologies linked to oxidative stress, wherein the method comprises the use of the combination according to claim 27, or a dermatological composition, comprising, as an active substance, said combination, with a pharmaceutically acceptable excipient.

40. A cosmetic method for treating or preventing skin aging, comprising the application to a healthy skin of a cosmetic composition according to claim 27.

41. The method of treatment according to claim 40, wherein said composition is applied from 1 to 3 times a day to cleansed skin.

42. The combination according to claim 27, wherein the tulip extract from the Tulipa Gesneriana family is a purple tulip extract.

43. The Combination according to claim 42, comprising or consisting of:
a purple tulip extract, at a rate of 0.1 to 5%, as a percentage by weight relative to the total weight of the combination, and
acetyl-tetrapeptide-2, at a rate of 0.005 to 0.05%, as a percentage by weight relative to the total weight of the combination, and
acetyl-hexapeptide-51 amide, at a rate of 0.005 to 0.05%, as a percentage by weight relative to the total weight of the combination.

44. The combination according to claim 42, comprising or consisting of:
a purple tulip extract, at a rate of 0.1 to 5%, as a percentage by weight relative to the total weight of the combination, and
acetyl-tetrapeptide-2, at a rate of 0.005 to 0.05%, as a percentage by weight relative to the total weight of the combination, and
acetyl-hexapeptide-51 amide, at a rate of 0.005 to 0.05%, as a percentage by weight relative to the total weight of the combination,
hyaluronic acid, at a rate of 1 to 25%, as a percentage by weight relative to the total weight of the combination.
